(19) United States
(12) Patent Application Publication
(10) Pub. No.: US 2007/0043368 A1
(43) Pub. Date: Feb. 22, 2007

(54) Distal humeral plate

(76) Inventors: Steven Lawrie, Buderim Queensland (AU); Russell Bourne, Buderim Queensland (AU)

Correspondence Address: MICHAEL MOLINS;MOLINS & CO., SUITE 5, LEVEL 6, 139 MACQUARIE ST, SYDNEY NSW, 2000, AU

(21) Appl. No.: 11/188,020

(22) Filed: Jul. 25, 2005

(51) Int. Cl. A61F 2/30

(52) U.S. Cl. 606/69

(57) ABSTRACT

A bone plate is provided for the distal humerus, the plate having a main shank portion with one or more screw holes. The main shank portion bends to form an off-axis shank portion that is free of screw holes. The off-axis portion twists 90 degrees to form a distal shank portion. A barrel formed on the distal shank portion allows for the insertion of a bone screw. The bone plate is thus affixed in two perpendicular planes by screwing the lower shaft portion to the humeral shaft and passing the bone screw through the distal humerus into the bevelled barrel.

### FIELD OF THE INVENTION

The invention relates generally to bone plates and more specifically to a plate and screw system used for fixation of fractures of the distal humerus.

### BACKGROUND AND RELATED ART

Certain types of fractures of the upper arm bones cannot be effectively healed without the use of a bone screw, pin, cable, wire or plate. The use of each of these is known to the prior art. Combinations of these are also used in an attempt to provide both internal and external securance of a fractured bone. Each of these, separately and in combination, is met with shortcomings in implementation, cost, and reliability.

In many cases, the use of a bone plate is the preferred treatment for a fracture. A bone plate is a sturdy plate typically constructed of metal that is custom shaped by a surgeon and affixed to a bone using screws or other fasteners. In cases involving the distal end of the humerus bone, current art includes the use of more than one bone plate.

Current plate solutions provide a plate with screw holes. The holes in the plate receive cooperating screws, affixing the plate to the bone. Once the bone is healed, the screws and plate are removed.

Current plates must generally be shaped to each individual bone before attaching. This is labour-intensive and time-consuming. In some cases, the shaping of the plate structurally weakens the metal of the plate.

### OBJECTS AND SUMMARY OF THE INVENTION

This invention relates to a plate and screw system used for fixation of fractures of the distal humerus, used to hold the broken bones of the humerus together as they heal. The device may be removed after the bones fuse. The plate terminates in a flat stem at one end and is curved to twist about and fit the lateral humeral condyle. The plate couples with a cannulated bone screw that is retained in an anti-rotation barrel.

It is one object of the present invention to provide a plate and screw system for fixation of fractures that does not require individual shaping of the plate to the arm. It is another object to provide a plate that is curved to fit the lateral humeral condyle without individual shaping. It is still another object to provide a method for fixing the plate in two perpendicular planes. This allows for increased strength of fixation and ease of use and avoids fixation to the acute angular projection of the lateral humeral shaft.

### BRIEF DESCRIPTION OF THE DRAWINGS

FIG. 1 is a top plan view of a bone plate in place on the distal humerus

FIG. 2 illustrates a top view of the bone plate of the invention

FIG. 3 illustrates a perspective view of the bone plate of the invention

FIG. 4 illustrates a perspective view of the bone plate of the invention

FIG. 5 is a cross sectional view of a cannulated bone screw retained in a bevelled barrel

FIG. 6 is a cross section through the barrel of FIG. 5

FIG. 7 is a side elevation of a bone plate

### DETAILED DESCRIPTION OF THE PREFERRED EMBODIMENTS

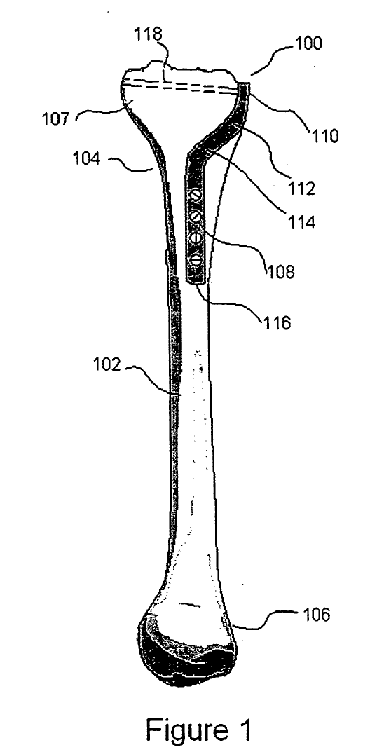

The bone plate of the present invention is adapted for fractures of the distal humerus. FIG. 1 illustrates a humerus bone 104 having a proximal end 106, a shaft 102, and a distal end 107. The bone plate of the invention 100, has a straight main shank portion 116 with several screw holes 108. The bone plate 100 leads into a curved or twisted portion 112 that accommodates the contours of the lateral humeral condyle, and terminates in a rounded end that further forms a barrel 216 for receiving a cannulated bone screw 118. Fixation screws 108 pass through the holes in the main shank 116. Because the plate wraps around the back of the humerus, twisting 90 degrees through the twisted portion 112, this allows a second fixation orthogonal to the shank 116 of the plate. The second fixation uses the cannulated bone screw 118. It is screwed directly into the bone, for example, by using a K wire passing through the cannula as a guide. Once located in the bone, the K wire is removed and the screw 118 inserted into the bevelled barrel 216 of the bone plate. As will be explained, the inserted end of the screw is bevelled so that when located in the barrel 216, the screw is prevented from rotating.

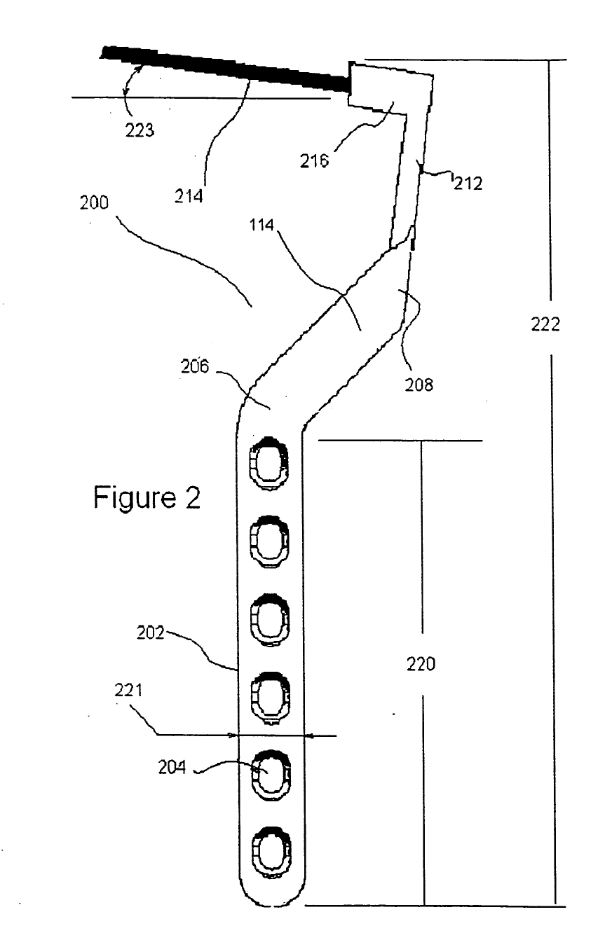

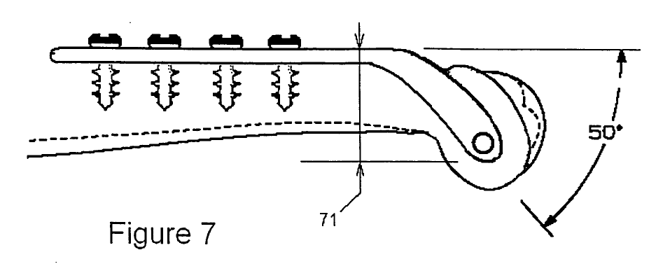

FIG. 2 illustrates a top view of the bone plate 200. The plate's main shank 202 is shown as provided with one or more oval, countersunk screw holes 204 along the shaft length. The holes are countersunk and sized to fit a cooperating screw (not shown). The shank has in intermediate portion comprising a bend 206 in the plane or the main portion that angles off at point above the last screw hole, the bend leading into the angled or off-axis shank portion 114. The off-axis shank portion 114 terminates in the twist portion 208 that leads into an upper or rotated shank portion 212. The upper shank 212 terminates in a bevelled barrel 216 into which the end of a cannulated bone screw 214 is inserted and retained As shown in this Figure, the length 220 of the main shank 202 is 72.52 mm measured from the proximal tip of the shank to the distal edge of the last screw hole. The width 221 of the main shank is 10.01 mm. The off-axis and distal portions are about the same width. The length 222 of the device measured from the distal tip of the barrel 216 to the proximal tip of the main shank is 130.31 mm. The angle 223 of the barrel and cannulated screw is 7.5 degrees from a line perpendicular to the long axis of the main shank 202. The width of the device (excluding the cannulated bone screw) and measured perpendicular to the long axis of the main shank is 33.27 mm. The interior tip of the barrel 216 is offset about 0.948 inches from the longitudinal centerline of the main shank. The depth of the device 71 (see FIG. 7), measured from the upper surface of the main shank to the lowest part extent of the distal shank portion is about 1.01 inches. These measurements should be seen as optimised but also representative and permit some scope for modification without departure from the scope or spirit of the invention.

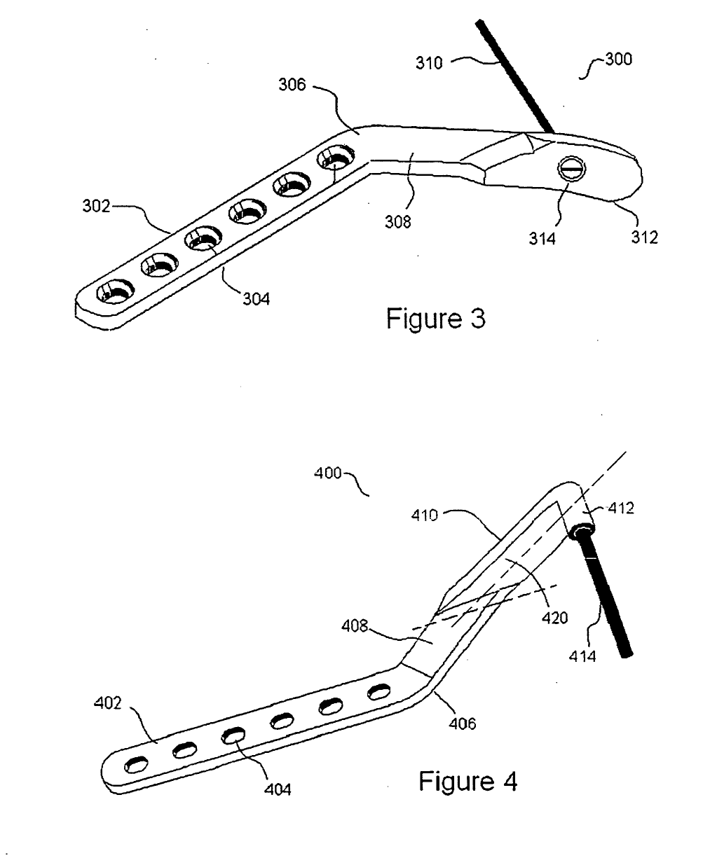

As shown in FIG. 3, a smooth, flat bend 306 leads to an off-axis shank portion 308 that is free of screw holes. The upper or rotated shank portion has a through opening 314 that receives a threaded fastener that engages the threaded end of the cannulated screw 310. The head of the fastener 316 is flush with the exterior surface of the upper shank portion 312.

As shown in FIG. 4 the upper shank portion 410 supports a barrel 412. The barrel extends form the upper shank at about 90 degrees and has an opening that receives the cannulated bone screw 414. The interior bore of the barrel forms a pair of bevels or flats that cooperate with matching bevels on the inserted, end of the screw. Once inserted, a threaded portion of the cannula can engage the fastener 316 (FIG. 3) and thus be drawn into and secured to the barrel, further rotation of the cannulated bone screw being prevented by the engagement of the bevels on the screw and the barrel. It can also be seen that the rotated or distal portion 410 of the shank is inclined 420 at an angle of 50 degrees with respect to a plane in which is located both the main shank portion 402 and the angled or off-axis portion 408 (see FIG. 7).

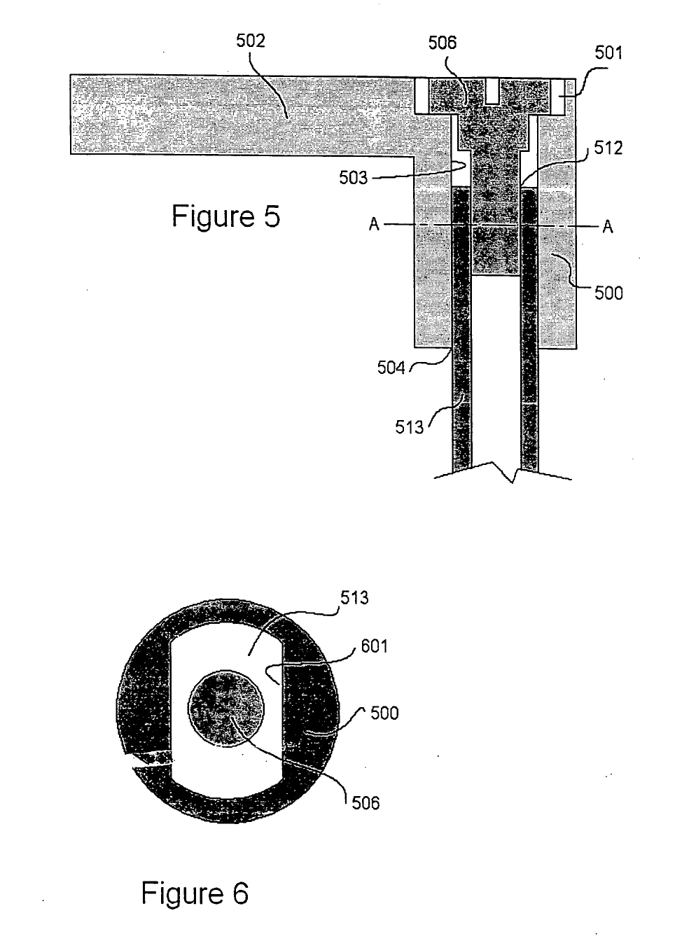

As shown in FIG. 5, the barrel 500 at the end of the rotated distal portion 502 of the shank features a through bore 503. The bore has a mouth 504 at the end of the barrel for receiving the screw 513 and a counter-bore 501 for receiving the threaded fastener 506. The fastener 506 enters the open end 512 of the cannulated bone screw 513 and engages cooperating threads in the cannula Rotation of the fastener 506 draws the screw into the barrel 500.

As shown in FIG. 6, internal bore of the barrel 500 is formed with opposing flats or bevels 601 that cooperate with flats or bevels formed on the exterior of the cannulated bone screw 513. Once the flats of the screw 513 and the barrel's bore are in engagement, the screw 513 is no longer free to rotate with respect to the barrel.

The bone plate is preferably fabricated from 316L stainless steel. The dimensions of the plate are in keeping with existing plates in the current art, using typical screw holes dimensions as known form DCP screw holes, thus allowing use of existing screws.

The bone plate as disclosed herein is anatomically specific to the anatomy of the lateral column of the distal humerus. The shape is determined from CT guided anatomical studies of the distal humerus, and considered unique. The bone plate is bent to match to the unique anatomy of the distal humerus and does not require any bending by a surgeon when applied.

As will be appreciated, the invention has been disclosed with reference to particular measurements and details of construction determined from anatomical studies intended to provide an optimal shape that does not need modification by the surgeon. These should be understood as relevant, optimised teachings but not necessarily as limitations to the scope or spirit of the invention.

What is claimed is:

1. A bone plate for the fixation of a fracture to the distal humerus, the plate comprising:
a main shank portion having at least one screw hole;
the main shank portion having a bend at one end that leads to an off-axis portion;
the off-axis portion terminating in a twist portion that leads to distal shank portion that is rotated with respect to the main shank portion;
the distal shank portion supporting a barrel;
the barrel having a bore, one open end of the bore adapted to receive a bone screw.

2. The bone plate of claim 1, wherein:
the twist portion acts to rotate the distal shank portion by about 90 degrees with respect to the main shank portion.

3. The bone plate of claim 1, wherein:
the distal shank portion is inclined with respect to a plane that includes the main and off-axis shank portions.

4. The bone plate of claim 1, wherein:
the barrel is inclined to a line that is perpendicular to a long axis of the main shank portion by about 7.5 degrees.

5. The bone plate of claim 3, wherein:
the distal shank portion is inclined by about 50 degrees.

6. The bone plate of claim 1, wherein:
the bore in the barrel further comprises one or more internal bevels.

7. The bone plate of claim 1, wherein:
the one or more bevels in the bore of the barrel engage with cooperating bevels formed on an inserted end of the bone screw to prevent the screw from rotating.

8. The bone plate of claim 7, wherein:
the bone screw is cannulated and the cannula, at least at the inserted end is internally threaded and there is provided a threaded fastener that enters the bore through the distal shank portion and engages the threads of the cannula.

9. The bone plate of claim 8, wherein:
the threaded fastener is received by a counter-bore so that it is flush with an external face of the distal shank portion.

10. The bone plate of claim 1, wherein:
the main, off-axis and distal shank portions are of approximately equal width.
